(19) United States
(12) Patent Application Publication
(10) Pub. No.: US 2014/0236540 A1
(43) Pub. Date: Aug. 21, 2014

(54) SIGNAL PROCESSING METHOD AND SIGNAL PROCESSING SYSTEM

(71) Applicant: INDUSTRIAL TECHNOLOGY RESEARCH INSTITUTE, CHUTUNG (TW)

(72) Inventors: Jun-Jie Chen, Taichung City (TW); Yen-Hsien Lee, Taipei City (TW); Hong-Dun Lin, Taipei City (TW)

(21) Appl. No.: 13/938,962

(22) Filed: Jul. 10, 2013

(30) Foreign Application Priority Data
Feb. 20, 2013 (TW) 102105867

(51) Int. Cl. A61B 5/00; A61B 5/04

(52) U.S. Cl. CPC A61B 5/7207; A61B 5/04017
USPC 702/194; 600/509

(57) ABSTRACT

A signal processing method and a signal processing system are provided. The signal processing method comprises the following steps. An original signal is provided. The original signal is gradually divided to be corresponding a plurality of stages. One high frequency signal and one low frequency signal whose frequency is lower than that of the high frequency signal are corresponding one of the stages. One of the low frequency signals corresponding to one of the stages is divided into another one of the high frequency signals and another one of the low frequency signals corresponding to the next one of the stages. An adjusting signal is obtained by filtering one of the low frequency signals out of another one of the low frequency signals.

This application claims the benefit of Taiwan application Serial No. 102105867, filed Feb. 20, 2013, the disclosure of which is incorporated by reference herein in its entirety.

### TECHNICAL FIELD

The disclosure relates in general to a processing method and a processing system, and more particularly to a signal processing method and a signal processing system.

### BACKGROUND

Along with the advance in technology, health status of human can be diagnosed according to varied measuring data. For example, an electrocardiography (ECG) signal can be used for diagnosing the physical fitness and the heart condition.

However, the accuracy of the signal measurement may be reduced due to the breathing signal and the electromyogram (EMG) signal. Currently, when a person is measured the ECG signal, the person is needed to be lain down for improving the accuracy of the signal measurement. As a result, the measurement of the ECG signal is limited to some particular situations and cannot be widely used.

On the other hand, when an industrial equipment measures some signals, the accuracy of the signal measurement may be interfered by the vibration of the industrial equipment. Therefore, it is needed to improve the accuracy of the signal measurement.

### SUMMARY

The disclosure is directed to a signal processing method and a signal processing system.

According to one embodiment, a signal processing method is provided. The signal processing method comprises the following steps. An original signal is provided. The original signal is gradually divided to be corresponding a plurality of stages. One high frequency signal and one low frequency signal whose frequency is lower than that of the high frequency signal are corresponding one of the stages. One of the low frequency signals corresponding to one of the stages is divided into another one of the high frequency signals and another one of the low frequency signals corresponding to the next one of the stages. An adjusting signal is obtained by filtering one of the low frequency signals out of another one of the low frequency signals.

According to another embodiment, a signal processing system is provided. The signal processing system comprises a providing unit, a dividing unit and a calculating unit. The providing unit is used for providing an original. The dividing unit is used for gradually dividing the original signal to be corresponding a plurality of stages. One high frequency signal and one low frequency signal whose frequency is lower than that of the high frequency signal are corresponding one of the stages. One of the low frequency signals corresponding to one of the stages is divided into another one of the high frequency signals and another one of the low frequency signals corresponding to the next one of the stages. The calculating unit is used for obtaining an adjusting signal by filtering one of the low frequency signals out of another one of the low frequency signals.

### BRIEF DESCRIPTION OF THE DRAWINGS

FIG. 1 shows a signal processing system.

FIG. 2 shows a flow chart of a signal processing method.

FIG. 3 is a waveform plot of an original signal.

FIG. 4 shows that the original signal is divided to be corresponding a plurality of stages.

FIG. 5 shows a plurality of high frequency signal and a plurality of low frequency signal.

FIG. 6 illustrates the original signal versus an adjusting signal.

FIG. 7A illustrates the original signal mixed 0 decibel noise versus an adjusting signal corresponding thereto.

FIG. 7B illustrates the original signal mixed 30 decibel noise versus an adjusting signal corresponding thereto.

FIG. 7C illustrates the original signal mixed 50 decibel noise versus an adjusting signal corresponding thereto.

FIG. 7D illustrates the original signal mixed 60 decibel noise versus an adjusting signal corresponding thereto.

FIG. 8A is a distribution diagram of the amplitude of the adjusting signal of FIG. 6 and a standard signal without any noise.

FIG. 8B illustrates the relationship between the amplitude and the quantity difference of the standard signal without any noise and the adjusting signal corresponding thereto.

FIG. 9 is a scatter plot of the standard signal without any noise and the adjusting signal.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

Preferred embodiments are disclosed below for elaborating the invention. The following embodiments are for the purpose of elaboration only, not for limiting the scope of protection of the invention. Besides, secondary elements are omitted in the following embodiments to highlight the technical features of the invention.

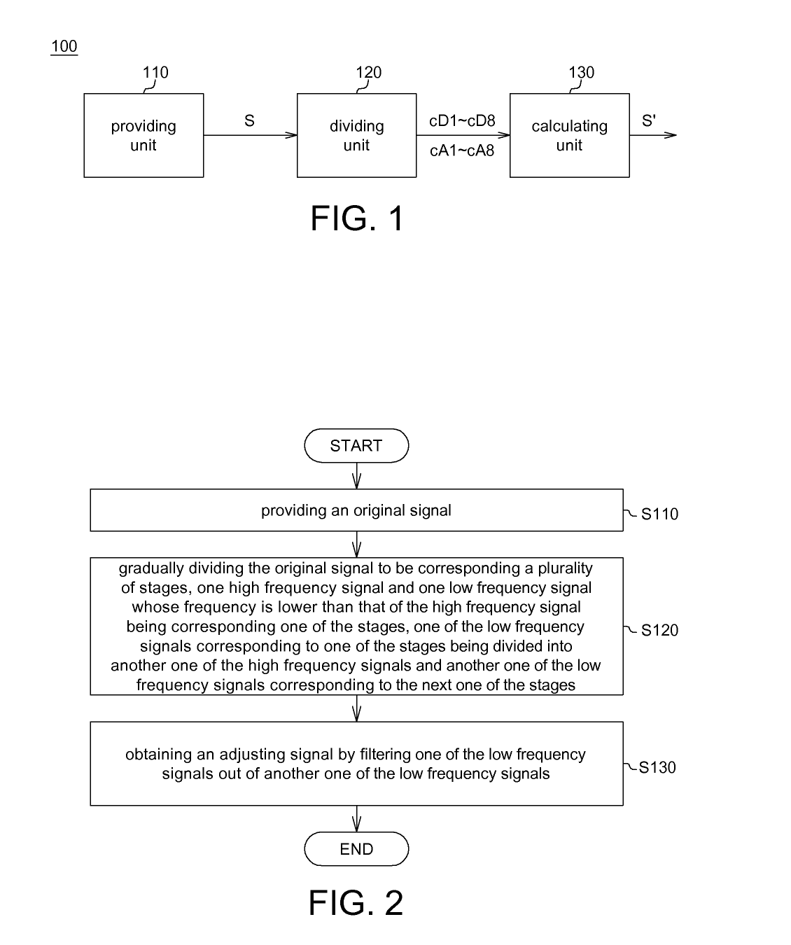

Please referring to FIG. 1, FIG. 1 shows a signal processing system 100. The signal processing system 100 includes a providing unit 110, a dividing unit 120 and a calculating unit 130. The signal processing system 100 can filter out noise with a particular frequency range for raising the signal to noise (SNR) by a wavelet coefficients analysis algorithm of the present embodiment.

The providing unit 110 is used for providing varied data. For example, the providing unit 110 may be a signal measuring device, a storage medium storing data or a transmission line connecting to a signal measuring device.

The dividing unit 120 is used for dividing varied data. For example, the dividing unit 120 may be a circuit board, a processing chip or a storage media storing a plurality of program codes.

The calculating unit 130 is used for performing varied calculating processes. For example, the calculating unit 130 may be a circuit board, a processing chip or a storage media storing a plurality of program codes. In one embodiment, the dividing unit 120 and the calculating unit 130 can be integrated into one circuit board, one processing chip or one storage media storing a plurality of program codes. In another embodiment, the dividing unit 120 and the calculating unit 130 can be two separated elements.

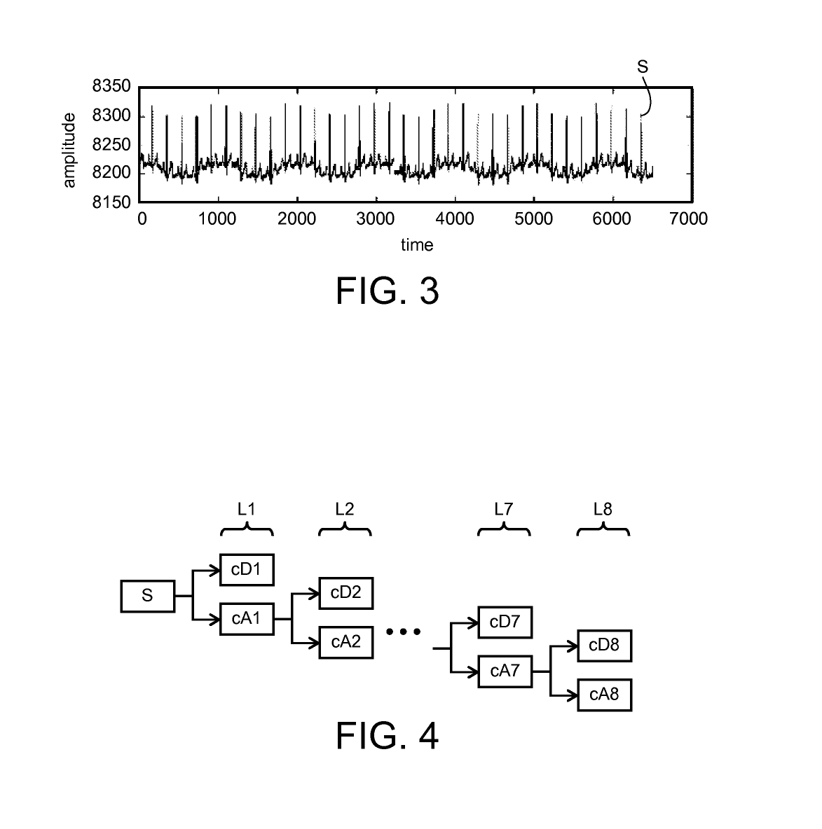

For clearly illustrating the operation of the signal processing system 100 of the present embodiment, a flow chart is provided. Please referring to FIG. 2, FIG. 2 shows a flow chart of a signal processing method. In step S110, an original signal S is provided by the providing unit 110. Please referring to FIG. 3, FIG. 3 is a waveform plot of the original signal S.

In one embodiment, the original signal S may be an electrocardiography (ECG) signal. When a person is exercising or rehabilitating, the measured ECG signal may contain noise signal caused by breathing, foot vibrating or body swinging. The particular noise signal can be filtered by the following steps. In another embodiment, the original signal S may be an audio signal broadcasted from a broadcasting equipment. The audio signal may contain noise signal caused by the vibration of a resonant film.

In step S120, the original signal S is divided to be corresponding a plurality of stages L1 to L8 by the dividing unit 120. Please referring to FIG. 4, FIG. 4 shows the stages L1 to L8. A high frequency signal cD1 and a low frequency signal cA1 whose frequency is lower than that of the high frequency signal cD1 are corresponding the stage L1. A high frequency signal cD2 and a low frequency signal cA2 whose frequency is lower than that of the high frequency signal cD2 are corresponding the stage L2. And so on, a high frequency signal cD8 and a low frequency signal cA8 whose frequency is lower than that of the high frequency signal cD8 are corresponding the stage L8.

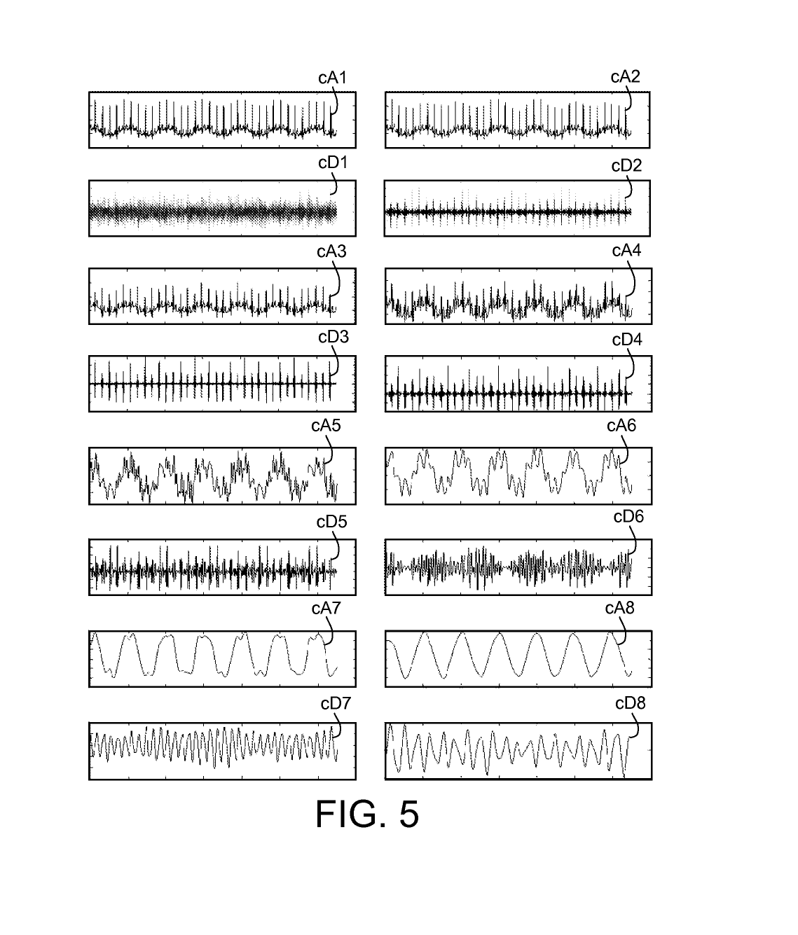

Please referring to FIGS. 4 and 5, FIG. 5 shows the high frequency signals cD1 to cD8 and the low frequency signals cA1 to cA8. At the stage L1, the original signal S is divided into the high frequency signal cD1 and the low frequency signal cA1. At the stage L2, the low frequency signal cA1 corresponding the stage L1 is divided into the high frequency signal cD2 and the low frequency signal cA2. At this time, high frequency noise signal is already filtered out from the low frequency signal cA2, and the low frequency signal cA2 may contain the breathing signal.

At stage L3, the low frequency signal cA2 corresponding the stage L2 is divided into the high frequency signal cD3 and the low frequency signal cA3. At this time, the QRS wave of the low frequency signal cA3 is started to be faded.

At stage L4, the low frequency signal cA3 corresponding the stage L3 is divided into the high frequency signal cD4 and the low frequency signal cA4. At this time, the QRS wave of the low frequency signal cA4 is already faded.

At the stage L5, the low frequency signal cA4 corresponding the stage L4 is divided into the high frequency signal cD5 and the low frequency signal cA5. At this time, the P wave, the T wave and the breathing character can be found in the low frequency signal cA5.

At the stage L6, the low frequency signal cA5 corresponding the stage L5 is divided into the high frequency signal cD6 and the low frequency signal cA6. At this time, the mix of the T wave and the breathing signal can be found in the low frequency signal cA6.

At the stage L7, the low frequency signal cA6 corresponding the stage L6 is divided into the high frequency signal cD7 and the low frequency signal cA7. At this time, the breathing signal can be found in the low frequency signal cA7.

At the stage L8, the low frequency signal cA7 corresponding the stage L7 is divided into the high frequency signal cD8 and the low frequency signal cA8. At this time, the residue of the breathing signal can be found in the low frequency signal cA8.

In the present embodiment, the ratio of the frequency range of the high frequency signals cD1 to cD8 to that of the corresponding the stages L1 to L8 are substantially identical. For example, the ratio can be 50%. The ratio of the frequency range of the low frequency signals cA1 to cA8 to that of the corresponding the stages L1 to L8 are substantially identical. For example, the ratio can be 50%. That is to say, at the stages L1 to L8, the high frequency signals cD1 to cD8 are gradually filtered out to remain the low frequency signals cA1 to cA8.

In step S130, an adjusting signal S′ is obtained by the calculating unit 130. The adjusting signal S′ is obtained by filtering one of the low frequency signals cA1 to cA8 out of another one of the low frequency signals cA1 to cA8.

In one embodiment, the adjusting signal S′ is obtained by filtering one of the low frequency signals cA1 to cA8 corresponding to one of the stages L1 to L8 with high level out of another one of the low frequency signals cA1 to cA8 corresponding to another one of the stages L1 to L8 with low level. The adjusting signal S′ can be obtained by the following equation (1).

S′=cAi−cAj, i<j and i,jε{1,2,3,4,5,6,7,8} (1)

For example, the adjusting signal S′ can be obtained by filtering the low frequency signal cA8 corresponding the stage L8 out of the low frequency signal cA2 corresponding to the stage L2. This adjusting signal S′ can be obtained by the following equation (2).

S′=cA2−cA8 (2)

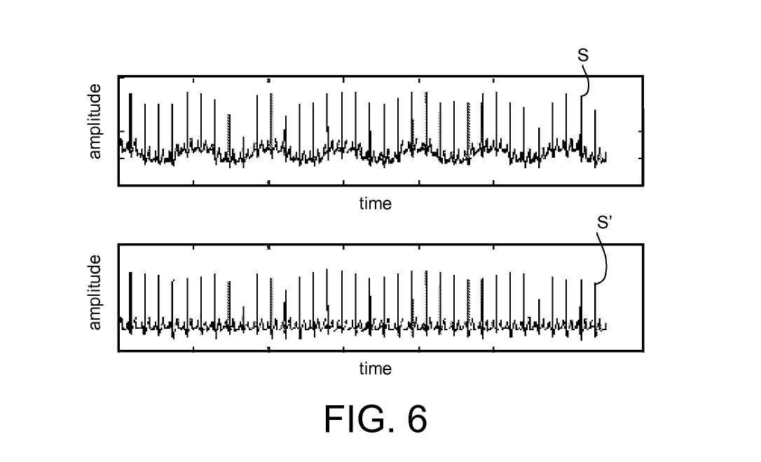

Please referring to FIG. 6, FIG. 6 illustrates the original signal versus the adjusting signal S′. The original signal S is fluctuated due to the noise caused by the breathing and the body moving. The adjusting signal S′ is stable because the noise caused by the breathing and the body moving is filtered.

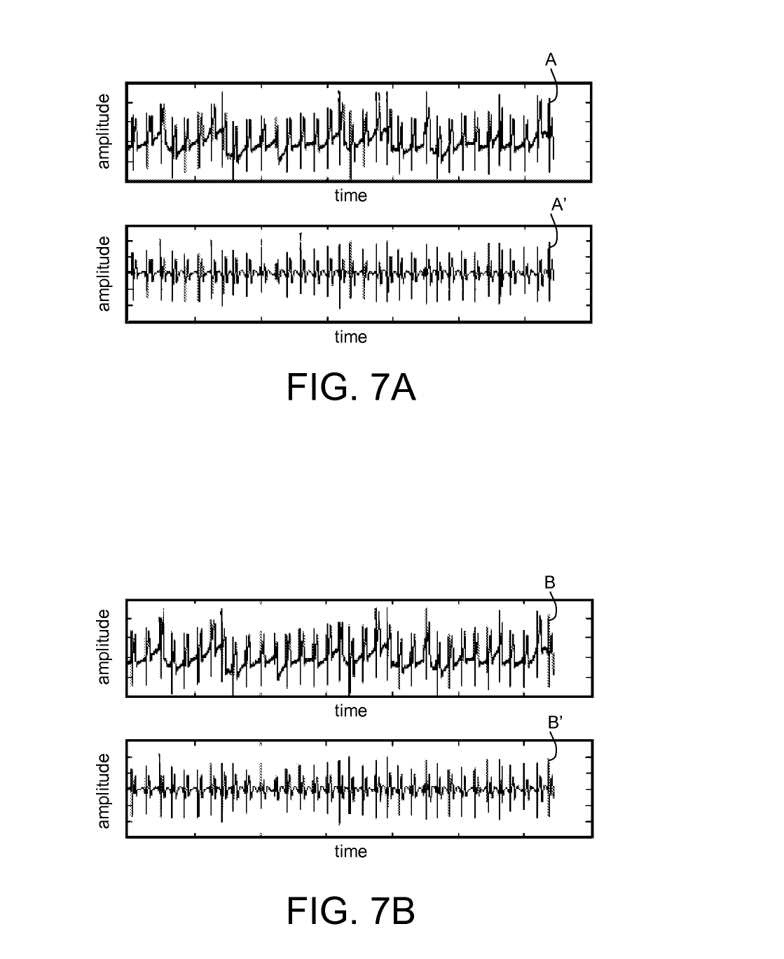

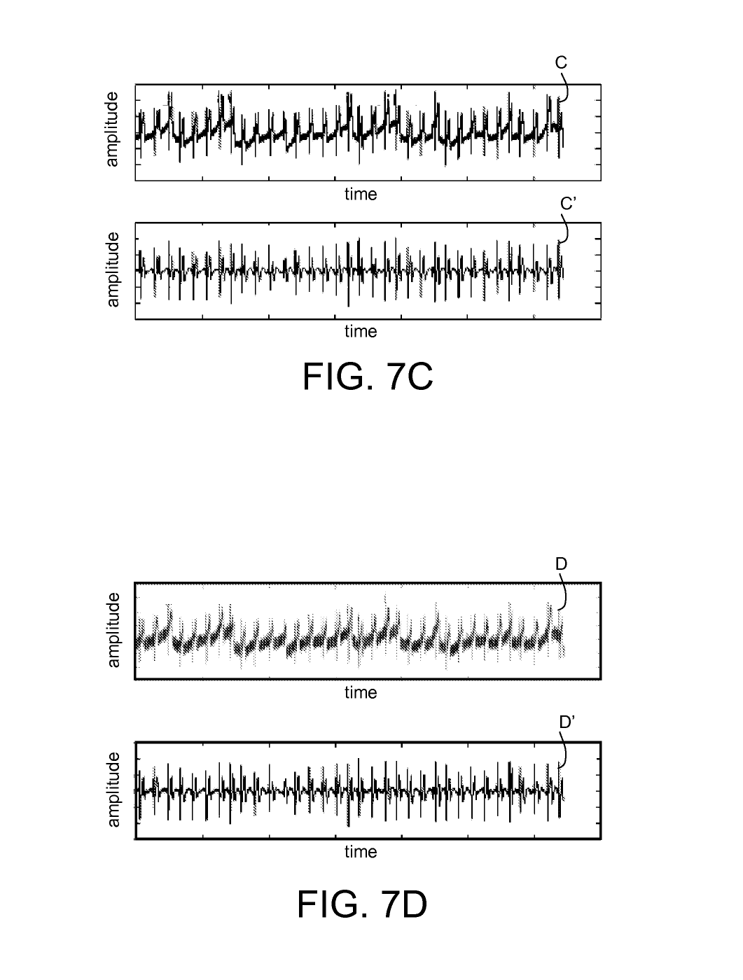

Please referring to FIGS. 7A to 7D, FIGS. 7A to 7D illustrate four original signals A, B, C and D mixed varied noise versus four adjusting signals A′, B′, C′ and D′ corresponding thereto. As shown in FIGS. 7A to 7D, the signal to noise ratio (SNR) can be improved by the signal processing method and the signal processing system 100 of the present embodiment.

Please referring to FIG. 7A, FIG. 7A illustrates the original signal A mixed 0 decibel noise versus the adjusting signal A′ corresponding thereto. The SNR of the adjusting signal A′ is improved by the signal processing method and the signal processing system 100 of the present embodiment.

Please referring to FIG. 7B, FIG. 7B illustrates the original signal B mixed 30 decibel noise versus the adjusting signal B′ corresponding thereto. The SNR of the adjusting signal B′ is improved by the signal processing method and the signal processing system 100 of the present embodiment.

Please referring to FIG. 7C, FIG. 7C illustrates the original signal C mixed 30 decibel noise versus the adjusting signal C′ corresponding thereto. The SNR of the adjusting signal C′ is improved by the signal processing method and the signal processing system 100 of the present embodiment.

Please referring to FIG. 7D, FIG. 7D illustrates the original signal D mixed 60 decibel noise versus the adjusting signal D′ corresponding thereto. The SNR of the adjusting signal D′ is improved by the signal processing method and the signal processing system 100 of the present embodiment.

As shown in the original signals A, B, C and D mixed varied noise, the original signal D which is mixed highest decibel noise is fluctuated obviously. No matter what the noise, the adjusting signals A′, B′, C′ and D′ obtained by the signal processing method and the signal processing system 100 do not have any obvious difference. That is to say, no matter what the noise, an adjusting signal having good quality can be obtained by the signal processing method and the signal processing system 100.

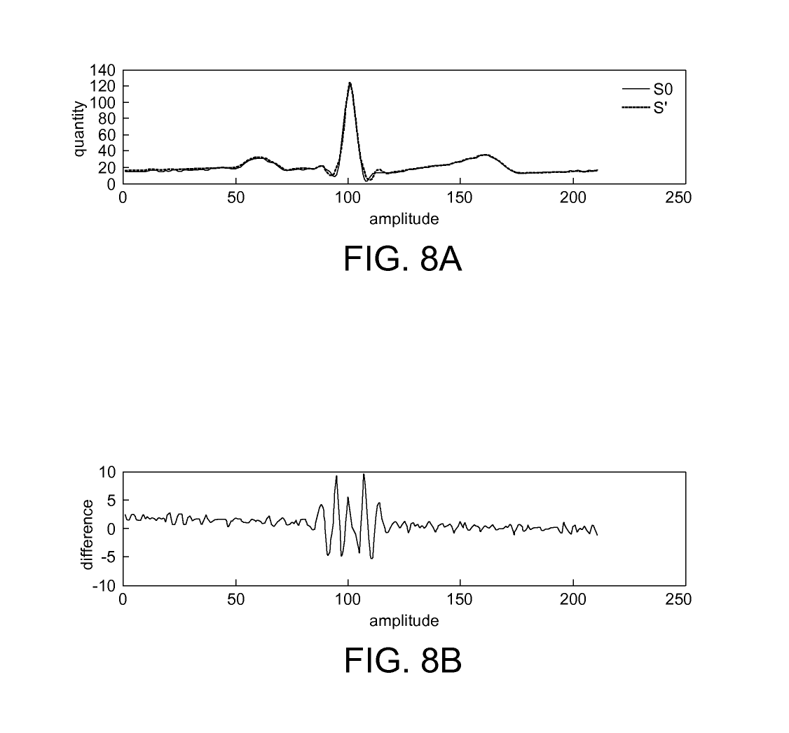

Please referring to FIG. 8A, FIG. 8A is a distribution diagram of the amplitude of the adjusting signal S′ of FIG. 6 and a standard signal S0 without any noise. As shown in FIG. 8A, the distribution of the amplitude of the adjusting signal S′ is similar to that of the standard signal S0.

Please referring to FIG. 8B, FIG. 8B illustrates the relationship between the amplitude and the quantity difference of the standard signal S0 without any noise and the adjusting signal S′ corresponding thereto. As shown in FIG. 8B, the quantity difference between the standard signal S0 and the adjusting signal S′ is small except that the amplitude thereof is at 100.

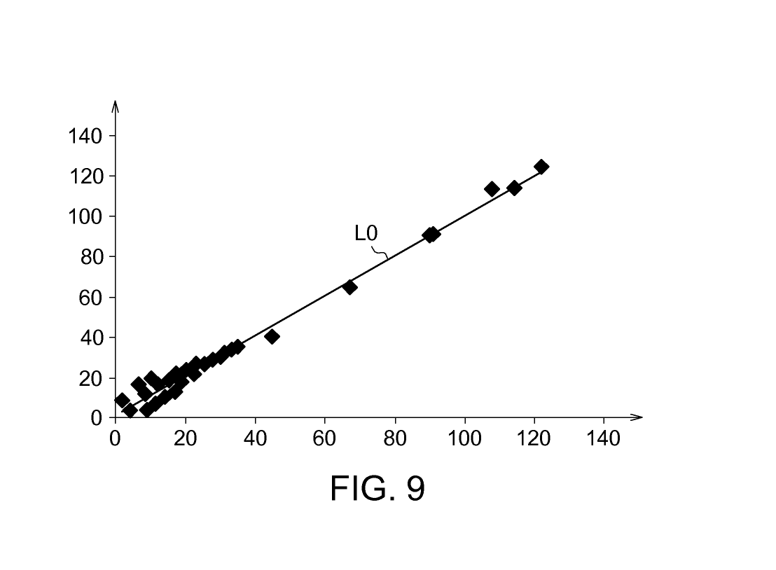

Please referring to FIG. 9, FIG. 9 is a scatter plot of the standard signal S0 without any noise and the adjusting signal S′. As shown in FIG. 9, the corresponding points between the standard signal S0 and the adjusting signal S′ are near to the regression line L0 and the R square R2 is substantially 0.9892. That is to say, the standard signal S0 and the adjusting signal S′ are high positive correlate.

As shown in FIGS. 8A to 9, the adjusting signal S′ obtained by the signal processing method and the signal processing system 100 of the present embodiment is quite similar to the standard signal S0. As such, even if the ECG signal is measured during exercise or rehabilitation, the ECG signal can be well adjusted.

The signal processing method and the signal processing system 100 of the present embodiment not only can be used for measuring the ECG signal but also can be used for measuring varied signals which are easily interfered with noise.

Moreover, the signal processing system 100 of the present embodiment can be a single device specialized for measuring a particular signal. Or, the signal processing system 100 of the present embodiment can be integrated into a home-care-equipment for providing more functions.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A signal processing method, comprising:
providing an original signal;
gradually dividing the original signal to be corresponding a plurality of stages, wherein one high frequency signal and one low frequency signal whose frequency is lower than that of the high frequency signal are corresponding one of the stages, and one of the low frequency signals corresponding to one of the stages is divided into another one of the high frequency signals and another one of the low frequency signals corresponding to the next one of the stages; and
obtaining an adjusting signal by filtering one of the low frequency signals out of another one of the low frequency signals.

2. The signal processing method according to claim 1, wherein the original signal is gradually divided to be corresponding the stages from low level to high level, and the adjusting signal is obtained by filtering one of the low frequency signals corresponding to one of the stages with high level out of another one of the low frequency signals corresponding to another one of the stages with low level.

3. The signal processing method according to claim 1, wherein the original signal is gradually divided to be corresponding the stages from level 1 to level n, and the adjusting signal is obtained by filtering the low frequency signal corresponding the stage with level 8 out of another low frequency signal corresponding to another stage with level 2.

4. The signal processing method according to claim 1, wherein the ratio of the frequency range of the high frequency signals to that of the corresponding the stages are substantially identical.

5. The signal processing method according to claim 1, wherein the ratio of the frequency range of the low frequency signals to that of the corresponding the stages are substantially identical.

6. The signal processing method according to claim 1, wherein the ratio of the frequency range of each high frequency signal to that of the corresponding the stage is 50%, and the ratio of the frequency range of each low frequency signal to that of the corresponding the stage is 50%.

7. The signal processing method according to claim 1, wherein the original signal is an electrocardiography (EGG) signal.

8. The signal processing method according to claim 7, wherein the another one of the low frequency signals which is filtered is a breathing signal.

9. A signal processing system, comprising:
a providing unit, used for providing an original signal;
a dividing unit, used for gradually dividing the original signal to be corresponding a plurality of stages, wherein one high frequency signal and one low frequency signal whose frequency is lower than that of the high frequency signal are corresponding one of the stages, and one of the low frequency signals corresponding to one of the stages is divided into another one of the high frequency signals and another one of the low frequency signals corresponding to the next one of the stages; and
a calculating unit, used for obtaining an adjusting signal by filtering one of the low frequency signals out of another one of the low frequency signals.

10. The signal processing system according to claim 9, wherein the original signal is gradually divided to be corresponding the stages from low level to high level, and the adjusting signal is obtained by filtering one of the low frequency signals corresponding to one of the stages with high level out of another one of the low frequency signals corresponding to another one of the stages with low level.

11. The signal processing system according to claim 9, wherein the original signal is gradually divided to be corresponding the stages from level 1 to level n, and the adjusting signal is obtained by filtering the low frequency signal corresponding the stage with level 8 out of another low frequency signal corresponding to another stage with level 2.

12. The signal processing system according to claim 9, wherein the ratio of the frequency range of the high frequency signals to that of the corresponding the stages are substantially identical.

13. The signal processing system according to claim 9, wherein the ratio of the frequency range of the low frequency signals to that of the corresponding the stages are substantially identical.

14. The signal processing system according to claim 9, wherein the ratio of the frequency range of each high frequency signal to that of the corresponding the stage is 50%, and the ratio of the frequency range of each low frequency signal to that of the corresponding the stage is 50%.

15. The signal processing system according to claim 9, wherein the original signal is an electrocardiography (EGG) signal.

16. The signal processing system according to claim 15, wherein the another one of the low frequency signals which is filtered is a breathing signal.
